(19) United States
(12) Patent Application Publication
(10) Pub. No.: US 2023/0078150 A1
(43) Pub. Date: Mar. 16, 2023

(54) DOUBLE-RESONANT COIL, ARRAY OF DOUBLE-RESONANT COILS, AND USE THEREOF

(71) Applicant: Forschungszentrum Juelich GmbH, Juelich (DE)

(72) Inventors: Suk Min Hong, Wuerselen (DE); Joerg Felder, Juelich (DE); Nadim Joni Shah, Juelich (DE); Chang-Hoon Choi, Juelich (DE)

(21) Appl. No.: 17/798,561

(22) PCT Filed: Mar. 4, 2021
(86) PCT No.: PCT/DE2021/000043

§ 371 Date: Aug. 10, 2022

(30) Foreign Application Priority Data
Mar. 13, 2020 (DE) 10 2020 001 654.5
Jan. 21, 2021 (DE) 10 2021 000 282.2

(51) Int. Cl. G01R 33/36; A61B 5/055

(52) U.S. Cl. CPC G01R 33/3642; G01R 33/3635; A61B 5/055

(57) ABSTRACT

A double-resonant coil includes a closed conductor loop divided into at least two segments that are each connected via a second connection element configured to be converted from an electrically conductive state to an electrically insulating state. The double-resonant coil further includes an inductor or a capacitor connected in series to a first connection element so as to allow excitation of the closed conductor loop in a dipole mode at one frequency and with a homogeneous power distribution at a second frequency.

### CROSS-REFERENCE TO PRIOR APPLICATIONS

This application is a U.S. National Phase application under 35 U.S.C. § 371 of International Application No. PCT/DE2021/000043, filed on Mar. 4, 2021, and claims benefit to German Patent Application No. DE 10 2020 001 654.5, filed on Mar. 13, 2020, and DE 10 2021 000 282.2, filed on Jan. 21, 2021. The International Application was published in German on Sep. 16, 2021 as WO/2021/180259 A1 under PCT Article 21(2).

### FIELD

The disclosure relates to a double-resonant coil and to an array of double-resonant coils and to the use thereof. The disclosure further relates to a coil arrangement for use as a transmitting and/or receiving coil in an MRT system or an MR/NMR spectroscopy system, which arrangement consists of one or more physical conductor structures on each of which two different power distributions can be excited. Each individual conductor structure thus has two different resonance frequencies. In addition, the disclosure relates to an MRT system or MR/NMR spectroscopy system having such a coil arrangement. Furthermore, the disclosure relates to the use of such a coil arrangement and to an array of double-resonant coils and to the use thereof.

### BACKGROUND

Magnetic resonance tomography, MRT for short, is an imaging method used in medical diagnostics by means of which structure, function and metabolism of tissues are displayed, especially in the form of sectional images. In magnetic resonance (MR)/nuclear magnetic resonance (NMR) spectroscopy, shifts of the nuclear magnetic resonance frequencies due to local variations of the underlying static magnetic field are shown as spectra or images. Due to the similarity of both methods, MRT systems and MR/NMR spectroscopy systems are hereinafter uniformly referred to as MR systems. The images captured using both systems are uniformly referred to as MR imaging.

In addition to the predominantly used MR imaging based on protons (hydrogen nuclei, ¹H), nuclear resonances of other nuclei having a nuclear spin other than zero can also be captured. These other nuclei are consolidated as X nuclei and comprise, e.g., metabolically active nuclei, such as sodium (²³Na) and phosphorus (³¹P). Due to the nuclear-specific value of the gyromagnetic constant, they have a different resonance frequency than the hydrogen nucleus. Due to the usually significantly lower concentration of X nuclei in biological tissue and the smaller resonance frequency, the received signal is lower in the case of X-nucleus MR imaging than in the case of MR imaging hydrogen nuclei by means of hydrogen nuclei. In the case of ultra-high field (UHF) MRT with a static field strength of ≥7 T, UHF MRT for short, a relatively high signal-to-noise ratio can nevertheless be achieved in the case of X-nucleus measurements compared to measurements at typical clinical field strengths of 1.5 T to 3 T. Nevertheless, x-nucleus measurements are usually carried out in combination with MR imaging by means of protons. On the one hand, this allows the superimposition of information from the X-nucleus measurement onto anatomical images that were captured by means of MR imaging of the hydrogen nucleus. On the other hand, overview images, so-called scout images, and adjustment layers, e.g., B₀ shimming, are carried out by means of ¹H measurements.

The efforts are therefore to generate coil arrangements that can excite and/or detect nuclear resonances of protons and X nuclei. In concrete terms, the coil arrangement must thus be able to emit and/or detect high-frequency electromagnetic fields on the ¹H resonance frequency and on at least one resonance frequency of an X nucleus. A double-resonant coil arrangement can accordingly emit and/or excite electromagnetic fields on the proton resonance frequency and the resonance frequency of a selected type of X nucleus.

A further effort, in particular for receiving the electromagnetic alternating field emitted by nuclear resonances, is to position the receiving coils as close as possible to the test volume and to use a high number of individual coils. Both maximize the signal-to-noise ratio of the received signal. In particular, it is of interest to use these elements advantageously in high-channel reception arrays, as are described, for example, in [1] in high-field MRT. Since coil arrangements are usually optimized for a specific test volume, for example for the human head in neuroimaging, the increase in the number of coil elements, which is also referred to as the channel count, inevitably leads to individual elements having smaller geometric dimensions. This in turn makes it more difficult to tune conventional surface coils, in particular for high resonance frequencies such as occur in UHF MRT. For example, the tuning capacitance of a circular surface coil having a free diameter of 85 mm is 1.4 pF at 7 T, 0.79 pF at 9.4 T, 0.5 pF at 11. 7 T and 0.35 pF at 14 T [2].

Numerous methods are known for the construction of dual-tuned or multiple-tuned MRT coil arrangements, such as are required for combined ¹H/X-nucleus measurements. All these methods aim to decouple protons and X-nucleus resonances. They can be categorized as follows:

Thus, decoupling by means of blocking circuits, e.g. dual-tuned birdcage resonators with passive blocking circuits, is usually described on alternating legs [3-5].

Furthermore, decoupling by means of PIN diodes is known from [6]. This can be employed, for example, when using two independent RF coils, which are alternately detuned.

Publications [7-8] show geometric decoupling, for example in the form of a “butterfly coil”.

Furthermore, modified resonator structures, e.g., birdcage resonators with additional end rings [9], are known.

Dipole and monopole antennas can be used in dual-tuned systems in combination with surface coils. If they are arranged in the center of the surface coil, a vanishing, coupled magnetic flux [10-12] results due to the magnetic fields of both conductor arrangements.

Folded dipole antennas are known from antenna technology. These can be connected at the dipole ends by an ohmic load and thus operated over a wide frequency range [13], also described in [14].

High-channel receiving coil arrangements for neuroimaging in MRT are described in [15-16].

Monoresonant, curved dipole antennas having an asymmetrical power distribution along the conductor (also referred to as “Loopoie”) are described in [17]. They are described as advantageous at a single resonance frequency in high-field MRT, because they can detect both partially conservative and non-conservative magnetic fields.

One possibility for realizing element coupling with the aid of a filter network between the individual elements is described in [18]. However, decoupling by means of filter networks is only possible at one frequency.

Publication [19] discloses a shortening of dipole antennas by means of inductors.

As already described above, small surface coils having a high channel count and high field strengths are practically untunable. To be sure, the capacitance can be brought to a realizable value for each individual capacitor by the series connection of two or more capacitors. However, the series connection leads to higher electrical losses due to a lower quality of the resonant circuit resulting from the series connection and thus leads to a deterioration of the signal-to-noise ratio of the MRT images. The tolerance of the total capacitance resulting from a series connection of individual capacitors is also problematic. This can exceed the value of the aforementioned total capacitance.

Double-resonant coils can be implemented by means of blocking circuits. The blocking circuit decouples additional capacitors from the coil at the ¹H frequency. The additional capacitors are therefore active only during the resonance of the coil at the X-nucleus frequency. This in turn results in the tuning on the ¹H frequency—as described above —remaining difficult even in double-resonant coils.

Alternative methods for generating double-resonant surface coils are often not feasible, or can only be realized with very great effort, due to the additionally required installation space in high-channel coil arrays. The additional effort includes, among other things, additional wires for supplying power to PIN diodes and a greater space requirement of the individual elements due to additional conductor tracks close to the coil elements.

Alternative antenna arrangements, as described in [17] have not yet been described in a double-resonant embodiment.

One possibility for realizing element coupling with the aid of a filter network between the individual elements is described in [18]. However, decoupling by means of filter networks is only possible at one frequency.

### SUMMARY

In an embodiment, the present disclosure provides a double-resonant coil. The double-resonant coil includes a closed conductor loop divided into at least two segments that are each connected via a second connection element configured to be converted from an electrically conductive state to an electrically insulating state. The double-resonant coil further includes an inductor or a capacitor connected in series to a first connection element so as to allow excitation of the closed conductor loop in a dipole mode at one frequency and with a homogeneous power distribution at a second frequency.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 depicts a simple embodiment of a coil according to the disclosure;

FIG. 2 depicts a coil according to the disclosure;

FIG. 3 depicts a coil according to FIG. 1 with connection of a reception chain;

FIG. 4 depicts measurement curves that represent an input reflection factor of a double-resonant coil according to the disclosure;

FIG. 5 depicts a proton scout image of an MRT phantom captured with a coil according to the disclosure;

FIG. 6 is a table providing values of the concentrated components of prototypes according to the disclosure; and

FIG. 7 is a table showing sensitivity of the double-resonant coil element compared to that of a similar monofrequency element on the X-nucleus resonance.

### DETAILED DESCRIPTION

The present disclosure provides a double-resonant coil that can be used as an element for a multi-channel structure in a coil arrangement. In addition to the ¹H resonance, each element should have a further resonance at the resonance frequency of a selected X-nucleus. In the case of a high number of channels and small physical dimensions, the double-resonant coil should be able to be easily tuned even at high field strengths (UHF-MRT). The additional ohmic losses, due to the inclusion of components relative to monofrequency coil designs, should remain low both on the ¹H resonance frequency and on the X-nucleus resonance frequency, so that the signal-to-noise ratio of the MRT images is similar to when monofrequency coils are used. The design should be possible by means of capacitors having commercially available tolerance values. The double-resonant coil or an arrangement constructed from coils of this type should be sufficient without additional space requirement for the supply of PIN diodes and a plurality of passive components. Protons and X-nucleus resonances should be adequately decoupled. It should be possible to decouple the individual coils of the coil array from one another for at least two frequencies.

With the double-resonant coil according to the present disclosure, it is now also possible to measure other X nuclei in addition to ¹H, wherein it can be used as an element for a multi-channel design. The double-resonant coil is easily tunable with a high channel count, in particular at high field strengths, which are usually ≥7 T. There are no additional electrical losses, for example on capacitors that are encumbered with losses due to the series connection. A high signal-to-noise ratio of MRT images and spectra remains ensured at both resonance frequencies. High tolerances caused by the series connection of a plurality of individual capacitors are avoided. The double-resonant coil or a coil array made of coils of this type can be realized without a greater space requirement of the individual elements caused by additional conductor tracks and/or components. Proton-nucleus and X-nucleus resonances on the individual coils and between the elements of a coil arrangement constructed from these elements can be decoupled.

In the following, the double-resonant coil according to the disclosure is described in its general form, without this being interpreted restrictively.

The disclosure is based on the consideration of using a physical structure both as a dipole antenna, namely with a dipolar power distribution at the ¹H resonance frequency, and as a conventional surface coil with homogeneous power distribution at the X-nucleus resonance frequency. At the same time, the coil element thus formed should be suitable for use in high-channel coil arrays. This requires, in particular, that it can have a geometrically compact design. If the ends of a folded dipole are connected to a capacitor (i.e., not ohmically), the structure thus formed is reminiscent of a conventional surface coil. By means of a connection element that can be converted from an electrically conductive state to an electrically insulating state, the geometry can be operated in a frequency-selective manner in a dipole mode, wherein the connection element is blocking for the proton frequency, and also in a surface coil mode with homogeneous power distribution, wherein the connection element has a different impedance than in the case of ¹H for the X-nucleus frequency. Since no capacitive tuning is necessary for proton frequency, tuning in the dipole mode takes place via conductor length, possibly also with the aid of methods for shortening or lengthening dipoles; the disadvantages of the prior art, in particular the demand for capacitors having small, non-realizable capacitance values, can be avoided.

Electromagnetic simulations of the arrangement have shown that the method described here can be realized at least up to field strengths of 14 T for antenna arrays having 32 channels for images of the human head.

According to the disclosure, a double-resonant coil, which is made of a closed conductor loop and is divided into at least two segments, is provided. The segments are each connected or bridged by a connection element, wherein the connection element can be converted from an electrically conductive state to an electrically insulating state. The segments connected via the connection elements form the conductor loop. The connection element is a means that is converted from an electrically conductive state to an electrically insulating state and is referred to below as a connection element for the sake of simplicity.

The conductor loop can consist of materials that are customary for this purpose, i.e., for example metal strips, wires or printed on a carrier material.

Both connection elements can be, for example, a blocking circuit, a PIN diode or an equivalent functional unit, wherein the blocking circuit is a preferred embodiment, because no active initiation is required for its operation.

The geometry of the conductor loop can be freely selected and can be selected such that it can be positioned well when used on a body part, for example on a head. It can be circular, oval, round or angular curved, polygonal, for example rectangular in elongated geometry, square, pentagonal, hexagonal, etc.

Both connection elements are preferably arranged within the conductor loop in such a way that they are located at positions that share the length of the conductor loop equally, because this makes possible a central feed of the dipole mode at the ¹H frequency.

An inductor or a capacitor is connected in series to a first connection element and can be used for adapting and/or for tuning the array to the resonance frequency of the X nuclei.

The conductor loop has points for feeding electrical power. These points are arranged on both sides of the first connection element. The distance to the first connection element is variable.

A further inductor or a further capacitor is connected in parallel to the arrangement made up of the first connection element and a capacitor or inductor connected in series. The capacitor connected in parallel or the inductor connected in parallel makes it possible for the coil to be adapted to the resonance frequency of the ¹H nucleus.

The other ends of the two segments of the conductor loop are connected by a second connection element, which is blocking at the ¹H frequency and thus forces a dipole-like power distribution at this frequency.

An inductor or a capacitor can be connected in series to this connection element. This has the advantage that a modification of the power distribution in the conductor loop can be carried out for the X nucleus.

Further capacitors and/or inductors or at least one further capacitor or at least one further inductor can be arranged along the conductor loop and can be used to shorten or extend the electrical length of the conductor arrangement for the ¹H and/or X-nucleus resonance.

Advantageously, the number of capacitors and/or inductors is determined according to the effective electrical length of the conductor loop. In this case, enough capacitors and/or inductors are arranged along the conductor loop to achieve a homogeneous power distribution for the X-nucleus frequency.

For example, a capacitor and/or an inductor can be inserted in the conductor loop, which is advantageously connected in series with the second connection element.

This ensures a homogeneous power distribution. However, a different position is also possible.

The capacitors in the conductor loop preferably have capacitances ≥1 pF, because otherwise there is the problem that small values that are less than 1 pF—which must be subject to tight tolerances and are correspondingly expensive—have to be used, and this should be advantageously excluded. In principle, the level of the capacitances according to upper values is not limited and is rather required by practical circumstances. The capacitances of the capacitors are expediently limited by the range of commercially available, concentrated, non-magnetic capacitors. This range is usually between 0.5 pF and 1 nF.

The conductor loop can furthermore be interrupted by at least one capacitor or at least one inductor. However, 2, 3, 4, 5, 6, 7., 8, 9 up to, for example, 20 capacitors and/or inductors can also be integrated into the conductor track. The number is generally open according to upper values and limited only by practical conditions. This makes it possible to shorten or lengthen the electrical length of the conductor loop. The position of the capacitors and/or inductors is freely selectable.

If these inductors or capacitors used to shorten or extend the electrical length of the conductor loop are to act only at a resonance frequency of the double-resonant coil, they can also be connected in series via a connection element, which can be converted from an electrically conductive state to an electrically blocking state. In this case, the parallel connection of a further capacitor or a further inductor that is active for both frequencies, is required, so that the conductor loop of the coil is not interrupted for a frequency.

The double-resonant coil can be arranged in at least two specimens for an array, which makes it possible for different body regions to be accessible to MRT measurement. A number of coils, for example 2 to 128 coils, can be used together for an array and are ideally arranged around the organ to be measured. A high number of individual double-resonant coils forms a high-channel array and leads to MRT images having a higher signal-to-noise ratio. In addition, the properties of the coil array with respect to methods of parallel imaging are improved by a high number of reception channels.

To decouple the coil elements at the X-nucleus resonance, known decoupling mechanisms can be used as means for decoupling coils due to the homogeneous power distribution. For the decoupling of the proton resonance, a filter network is inserted between the inputs of adjacent coils. In order for the filter network not to change the adaptation of the coil in the X-nucleus resonance, each filter element must be provided with a connection element that can be converted from an electrically conductive state to an electrically blocking state.

The individual coils should preferably have a small size in order to be able to arrange as many elements as possible on a given surface. The diameters are limited downward by the desired immersion depth of the electromagnetic field into the test volume and upward by the minimum desired number of elements of the array. Typical diameters in the field of neuroimaging are, for example, between 30 cm and 10 mm.

With the double-resonant coil according to the disclosure, reliable tuning can also be performed with small dimensions of the coils and high magnetic fields of ≥7 T. The coil or the array of coils can be used at high field strengths of, for example, 7 T to 21 T for obtaining very good MRT images. The field strengths in which the coils can be used are generally open according to upper values and only limited by the practical feasibility of magnets having corresponding field strengths.

The double-resonant coil or the array of double-resonant coils can be used for ¹H nuclei and X-nuclei. The X nuclei used can be all nuclei that have a nuclear spin other than zero and can therefore be used in MRT imaging, for example, but not limited to ²H, ⁷Li, ¹³C, ¹⁷O, ¹⁹F, ²³Na, ³¹P, ³⁵CI and ³⁹K.

In an advantageous embodiment, the double-resonant coil has two further connection elements that block the double-resonant coil during transmission with a different, additional transmission coil. These connection elements prevent a current flow during the transmission at the two resonance frequencies, namely that of the ¹H and that of the X-nucleus. Both connection elements can be PIN diodes or antiparallel PN diodes. The design of the connection elements is known to a person skilled in the art.

In an advantageous embodiment of the double-resonant coil, the points for feeding electrical power can be used for both resonance frequencies. For this purpose, when narrowband pre-amplifiers are used, such as are usually used for optimizing the SNR in MRT, a frequency-selective division of the received signals is necessary. The methods are known to a person skilled in the art.

The disclosure also relates to an array of the double-resonant coils. In the array, the double-resonant coils are arranged so as to be arranged around a body part, in particular around a head.

The double-resonant coils or an array thereof can be used in NMR/MR spectroscopy or MR tomography.

The figures show the double-resonant coil according to the disclosure and its designs in schematic form.

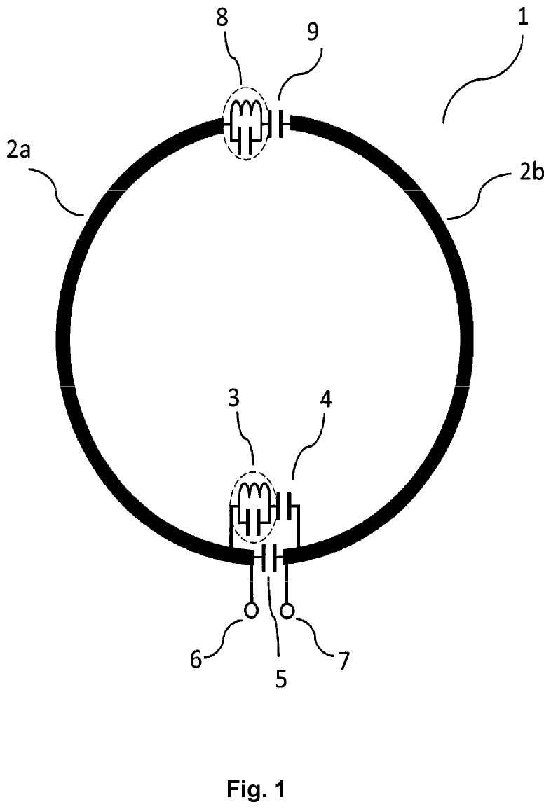

FIG. 1 shows a simple embodiment of the double-resonant coil having a conductor loop 1 made up of two segments 2a, 2b. It has a first connection element designed as a blocking circuit 3, to which a capacitor 4 is connected in series. A capacitor 5 is connected in parallel to the blocking circuit 3 connected in series to the capacitor 4. Furthermore, inputs 6, 7 for feeding electrical power are arranged on both sides of the capacitor 4 with a series-connected blocking circuit 3. The two ends of the segments 2a, 2b are connected via a second connection element designed as an oscillating circuit 8. A further capacitor 9 is connected in series to the second connection element.

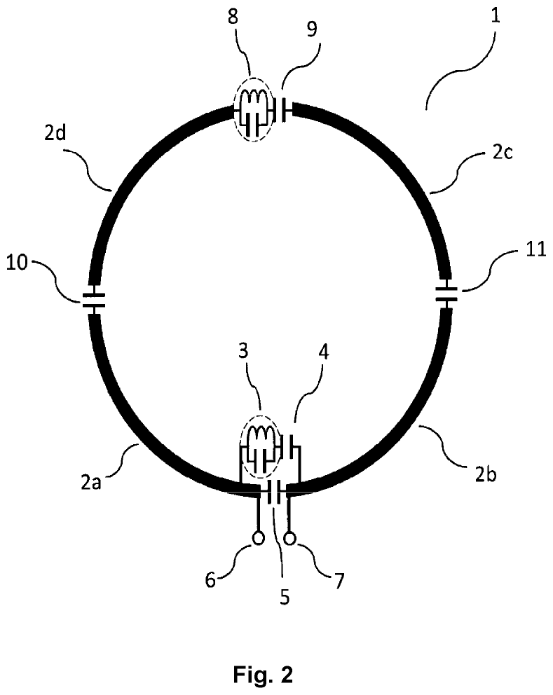

FIG. 2 shows an embodiment of the double-resonant coil. In this embodiment, identical device characteristics have the same reference signs. In this embodiment, there are four segments 2a, 2b, 2c, 2d in the place of the two segments 2a, 2b, of which, in each case, 2 of them are connected by capacitors 10, 11.

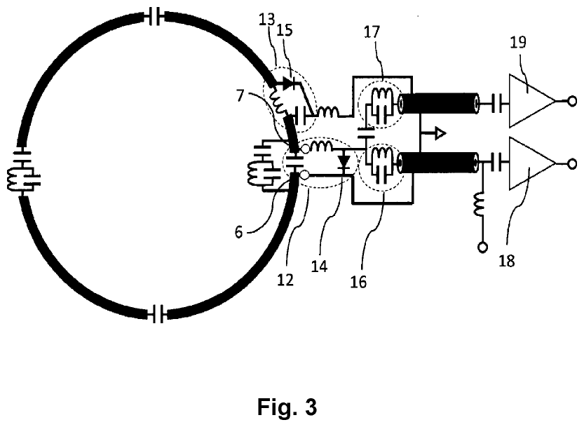

FIG. 3 shows a possibility of connecting the coil from FIG. 1 to a reception chain for the two resonance frequencies. In this embodiment, identical device characteristics have the same reference signs. Furthermore, two additional connection elements are designed to be arranged in FIG. 3 as detuning circuits 12, 13, which, when the coil according to FIG. 1 is used as a receiving element, allow detuning at both resonance frequencies during the transmission pulse. The connection elements, embodied as detuning circuits 12 and 13, can be switched by means of PIN diodes 14 and 15. The frequency diplexer for dividing the received signal tapped for both resonance frequencies at the feed points 6, 7 is formed from the two resonant circuits 16 and 17. These guide the received signal to the narrowband pre-amplifiers 18 and 19.

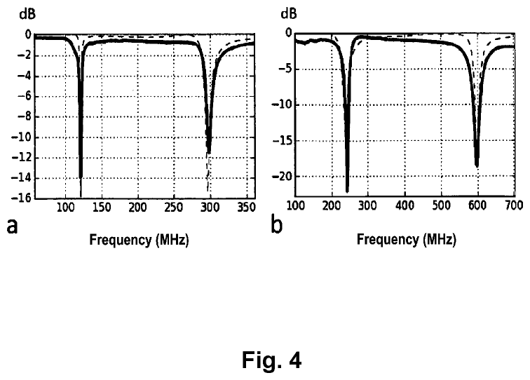

FIG. 4 shows two measurement curves a and b, which represent the input reflection factor of a double-resonant coil. Illustration a shows the input reflection factor for a coil that can be used at a static field strength of 7 T for protons and phosphorus (³¹P). Illustration b shows an embodiment for 14 T. In both figures, the measured values on a prototype are shown as a solid line and those from a 3D field simulation as a dashed curve.

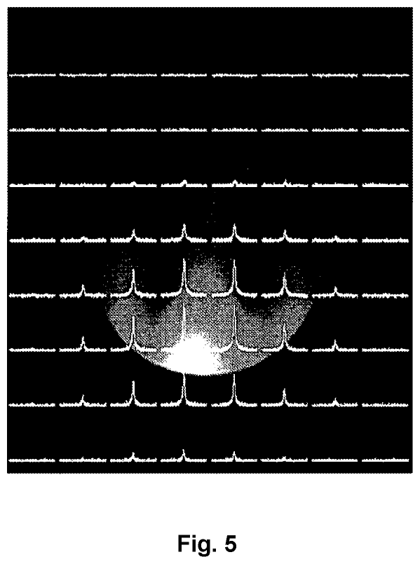

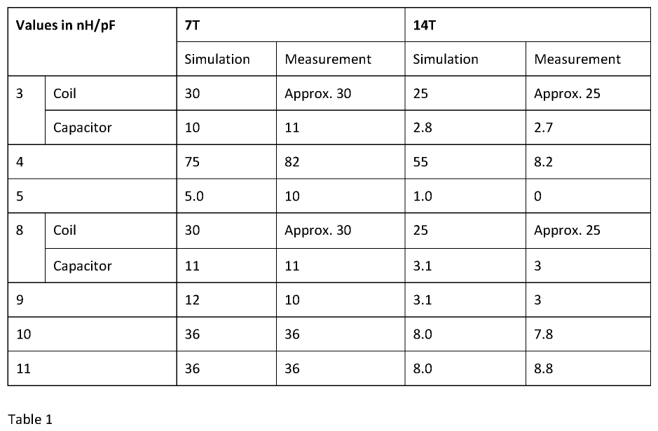

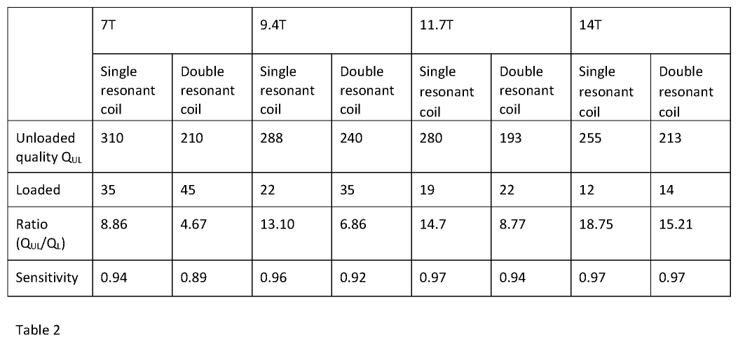

FIG. 5 shows a proton scout image of an MRT phantom captured with a coil according to the disclosure. Localized ³¹P spectra, which are also captured with the coil, are superimposed.

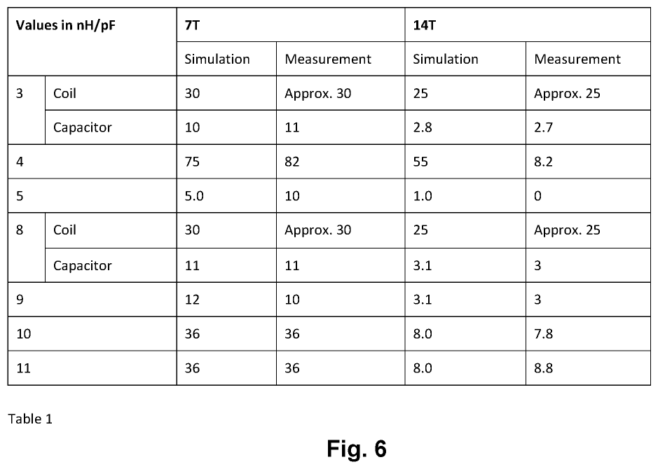

FIG. 6 is a table providing values of the concentrated components of prototypes according to the disclosure.

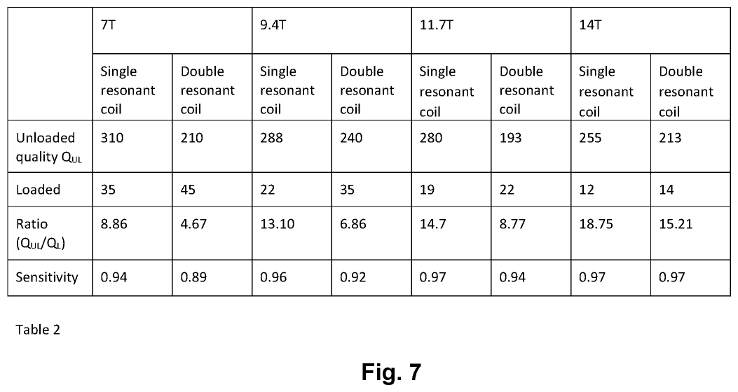

FIG. 7 is a table showing sensitivity of the double-resonant coil element compared to that of a similar monofrequency element on the X-nucleus resonance.

### EXAMPLE

For a high-channel reception array, an element diameter of approximately 85 mm, for example, can be suitable. Starting from this dimension, a dipole having a circular curvature can be created, the resonance of which corresponds to approximately 490 MHz due to the conductor length. FIG. 1 (without connection elements 3, 8 and without capacitors 4, 9) shows the arrangement of the dipole in which additionally concentrated components, in this case capacitors, for tuning and frequency adjustment have been introduced. The required concentrated components can also be coils or can be replicated by analogous, non-concentrated elements, depending on the ¹H resonance frequency.

For the operation as an X-nucleus surface coil, the blocking circuit 8 must be tuned to the proton frequency. The X-nucleus resonance is then set via 9.

It can be advantageous to insert a further blocking circuit 3 and a further capacitor 4 in the circuit. This simplifies the coordination and adaptation of the X-nucleus resonance when 3 in turn has a high impedance (resonance) at the proton frequency.

A simulation model and the prototype of the individual coil arrangement were used to determine the input reflection factor of the arrangement. The measured and simulated resonances (¹H/³¹P) are shown in FIG. 4—for 7 T in FIG. 4a and for 14 T in FIG. 4b. The inductance and capacitance values used for both frequencies are shown in Table 1. Table 2 shows that the X-nucleus coil loses only a little sensitivity compared to a monofrequency arrangement, so that the dual-tuned arrangement has only small SNR losses compared to a coil that is only tuned to the X-nucleus frequency.

A validation of the arrangement in MRT is shown in FIG. 5. In addition to the protons image, captured with a single coil element, the localized phosphorous spectrum thus obtained is also shown therein.

A slightly modified version of the coil is shown in FIG. 3. In this case, the ¹H and ³¹P reception channels are discharged via a common tap on the coil conductor. This arrangement advantageously enables the detuning of the receiving element by means of PIN diodes, both during the excitation at the ¹H and at the ³¹P resonance frequency.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### LIST OF REFERENCES

[1] B. C. Rowland et al., “Whole brain P MRSI at 7T with a dual-tuned receive array”, Magnetic Resonance in Medicine, vol. 0, no. 0.

[2] G. C. Wiggins, C. Triantafyllou, A. Potthast, A. Reykowski, M. Nittka, and L. L. Wald, “32-channel 3 Tesla receive-only phased-array head coil with soccer-ball element geometry”, Magn Reson Med, vol. 56, no. 1, pp. 216-223, July 2006.

[3] Shen G X, Wu J f, Boada F E, Thulborn K R. Experimentally verified, theoretical design of dual-tuned, low-pass birdcage radiofrequency resonators for magnetic resonance imaging and magnetic resonance spectroscopy of human brain at 3.0 Tesla. Magn. Reson. Med. 1999;41:268-275. doi: 10.1002/(SICI)1522-2594(199902)41: 2<268::AID-MRM9>3.0.CO; 2-G.

[4] Meyerspeer M, Roig E S, Gruetter R, Magill A W. An improved trap design for decoupling multinuclear RF coils. Magn. Reson. Med. 2013:n/a-n/a. doi: 10.1002/mrm.24931.

[5] Dabirzadeh A, McDougall M P. Trap design for insertable second-nuclei radiofrequency coils for magnetic resonance imaging and spectroscopy. Concepts Magn. Reson. Part B Magn. Reson. Eng. 2009;35B: 121-132. doi: 10.1002/cmr.b.20139.

[6] Ha S, Hamamura M J, Nalcioglu O, Muftuler L T. A PIN diode controlled dual-tuned MRI RF coil and phased array for multi nuclear imaging. Phys. Med. Biol. 2010;55:2589-2600. doi: 10.1088/0031-9155/55/9/011.

[7] Bottomley P A, Hardy C J, Roemer P B, Mueller O M. Proton-decoupled, overhauser-enhanced, spatially localized carbon-13 spectroscopy in humans. Magn. Reson. Med. 1989;12:348-363. doi: 10.1002/mrm.1910120307.

[8] Adriany G, Gruetter R. A half-volume coil for efficient proton decoupling in humans at 4 tesla. J. Magn. Reson. San Diego Calif. 1997 1997;125:178-184. doi: 10.1006/jmre.1997.1113.

[9] Potter W m., Wang L, McCully K k., Zhao Q. Evaluation of a new 1H/31P dual-tuned birdcage coil for 31P spectroscopy. Concepts Magn. Reson. Part B Magn. Reson. Eng. 2013;43:90-99. doi: 10.1002/cmr.b.21239.

[10] Shajan G, Mirkes C, Buckenmaier K, Hoffmann J, Pohmann R, Scheffler K. Three-layered radio frequency coil arrangement for sodium MRI of the human brain at 9.4 Tesla. Magn. Reson. Med. 2015:n/a-n/a. doi: 10.1002/mrm.25666.

[11] Yan X, Wei L, Xue R, Zhang X. Hybrid Monopole/Loop Coil Array for Human Head MR Imaging at 7 T. Appl. Magn. Reson. 2015:1-10. doi: 10.1007/s00723-015-0656-5.

[12] Yan X, Xue R, Zhang X. A monopole/loop dual-tuned RF coil for ultrahigh field MRI. Quant. Imaging Med. Surg. 2014;4:225-231.

[13] Elmer R. Bush, Broad bandwidth folded dipole antenna, U.S. Pat. No. 4,423,423A

[14] An Experimental All-Band Nondirectional Transmitting Antenna by Gil L. Countryman, W1RBK, (W3HH), QST, June 1949, p. 54.

[15] Wiggins, G. C., Polimeni, J. R., Potthast, A., Schmitt, M., Alagappan, V. and Wald, L. L. (2009), 96-Channel receive-only head coil for 3 Tesla: Design optimization and evaluation. Magn. Reson. Med., 62: 754-762. doi:10.1002/mrm.22028

[16] Keil, B., Blau, J.N., Biber, S., Hoecht, P., Tountcheva, V., Setsompop, K., Triantafyllou, C. and Wald, L.L. (2013), A 64-channel 3T array coil for accelerated brain MRI. Magn Reson Med, 70: 248-258. doi: 10.1002/mrm.24427

[17] K. Lakshmanan, M. Cloos, and G. C. Wiggins, “Circular dipole and surface coil loop structures and methods for using the same”, US20150137815A1, 21 May 2015.

[18] I. R. O. Connell and R. S. Menon, “Shape Optimization of an Electric Dipole Array for 7 Tesla Neuroimaging”, in IEEE Transactions on Medical Imaging, vol. 38, no. 9, pp. 2177-2187, September 2019. doi: 10.1109/TMI.2019.2906507

[18] U.S. Pat. No. 2,229,865 “Radio antenna system”

1. A double-resonant coil, comprising:
a closed conductor loop divided into at least two segments that are each connected via a second connection element configured to be converted from an electrically conductive state to an electrically insulating state; and
an inductor or a capacitor connected in series to a first connection element so as to allow excitation of the closed conductor loop in a dipole mode at one frequency and with a homogeneous power distribution at a second frequency.

2. The double-resonant coil according to claim 1, wherein the first or the second connection element is a blocking circuit or a PIN diode.

3. The double-resonant coil according to claim 1, wherein the first and second connection elements share the conductor loop equally.

4. The double-resonant coil according to claim 1, wherein a capacitor or an inductor is connected in parallel to the first connection element.

5. The double-resonant coil according to claim 1, further comprising a feed for electrical power.

6. The double-resonant coil according to claim 5, wherein the feed for electrical power is connected on both sides of the first connection element.

7. The double-resonant coil according to claim 1, wherein a capacitor or an inductor is connected in series on the second connection element.

8. The double-resonant coil according to claim 1, wherein at least one further capacitor and/or at least one further inductor, which can be used to shorten or extend the electrical length of the conductor arrangement on a ¹H and/or X-nucleus resonance, is arranged along the conductor loop.

9. The double-resonant coil according to claim 1, wherein capacitors have capacitances ≥1 pF up to 1000 pF.

10. The double-resonant coil according to claim 1, further comprising two further connection elements configured to block the double-resonant coil during transmission with a further transmission coil.

11. The double-resonant coil according to claim 10, wherein a plurality of coils is interconnected to form an array.

12. An array of double-resonant coils according to claim 1.

13. The array according to claim 12, further comprising a decoupler configured to decouple individual double-resonant coils.

14. The array according to claim 12, the array having a quantity of 2 to 128 coils.

15. The array according to claim 12, wherein the coils are arranged in such a way that they at least partially surround a head.

16. The use of a coil according to claim 1 in magnetic resonance spectroscopy or magnetic resonance tomography.
